(19) United States
(12) Patent Application Publication
(10) Pub. No.: US 2022/0105039 A1
(43) Pub. Date: Apr. 7, 2022

(54) Use of Nanoparticles to Stabilize and Preserve the Bioactivity of Proteins and Peptides

(71) Applicant: University of Pittsburgh - Of the Commonwealth System of Higher Education, Pittsburgh, PA (US)

(72) Inventors: Xinyan Cui, Wexford, PA (US); Kevin M. Woeppel, Pittsburgh, PA (US)

(21) Appl. No.: 17/605,718

(22) PCT Filed: Apr. 23, 2020
(86) PCT No.: PCT/US2020/029544

§ 371 Date: Oct. 22, 2021

Related U.S. Application Data
(60) Provisional application No. 62/837,223, filed on Apr. 23, 2019.

(51) Int. Cl. A61K 9/16; A61K 9/00; A61B 5/1486; A61N 1/05

(52) U.S. Cl. CPC A61K 9/1658; A61K 9/0024; A61B 2562/125; A61B 5/1486; A61N 1/0565; A61K 9/1611

(57) ABSTRACT

Provided herein are methods of making and using storage-stable protein-coated articles that retain activity after drying. Also provided herein are kits comprising storage-stable protein-coated articles that retain activity after drying.

### CROSS REFERENCE TO RELATED APPLICATIONS

This application claims the benefit of U.S. Provisional Patent Application No. 62/837,223 filed Apr. 23, 2019, which is incorporated herein by reference in its entirety.

Maintaining the bioactivity of surface-bound protein during storage and sterilization is a common challenge for immunoassays, protein arrays, enzyme-based biosensors, and peptide functionalized medical implants. Among the methods explored, dry storage is preferred over wet because it makes packaging and delivery of delicate micro-devices easier. Wet condition also increases the chance of hydrolysis degradation of both the protein and the linkage between protein and substrate, therefore, should be avoided. Freeze-drying (lyophilization) and low temperature storage is a common practice in biochemistry and protein pharmaceutics for prolonging the activity of free proteins. Inspired by this, several groups have investigated the effectiveness of freeze-drying for prolonging the storage life of surface-immobilized proteins, and they found that the methods of surface immobilization, freeze drying condition, storage temperature, humidity, and duration impact the bioactivity of the surface bound proteins. To further improve stability prior to freeze-drying, a protective overcoat composed of layer-by-layer-deposited mannitol and polyelectrolytes has been reported to be effective in protecting vaccine antigen. Alternatively, polyethylene glycol gel may be applied to preserve the bioactivity of the underlying protein upon freeze-drying and storage, a method that has shown effectiveness in preserving and even increasing the bioactivity of glucose oxidase in glucose sensors.

In further detail, fabrication of electrodes, implants, sensors, and prosthetics is most often performed via inorganic or non-biologic materials. Materials such as silicon, silicone, stainless steel, noble metals, and many plastics are widely utilized due to their ease of microfabrication, availability, mechanical properties, and lack of toxicity. However, the inert nature of these materials minimizes their ability controllably interface with biological tissues, thereby limiting the capacity for tissue regeneration, sensing, and potentially resulting in device failure due to uncontrolled inflammation and scar tissue encapsulation. In order to impart biological activity to both implantable and non-implantable devices, a wide variety of surface modifications have been explored.

Immobilization of proteins and peptides is a particularly attractive option as many of these molecules are created for the sole purpose of adhesion, regeneration, detection, and catalysis at physiological conditions. Due to their high bioactivity and low threshold concentration of activity, protein surface modifications are a powerful tool, through which broad range of functionalities can be created without demanding changes to the bulk of the device. Direct immobilization of proteins has allowed otherwise inactive materials to directly control tissue reactions, become chemical sensors, and to serve as a scaffold for regenerative medicine.

However, protein lifetimes in ambient or biological conditions are limited. The highly-controlled tertiary structure of proteins is prone to denaturing, and proteases can attack the amide bonds holding the molecule together. It is, therefore, imperative to protect and stabilize the bound molecules if there is any desire for chronic activity.

Preservation of activity of bioactive surfaces in physiological and ambient dry conditions is a matter of commercial viability and opens the door for broader and more robust supply chains. Practical solutions are needed for improving the lifetime and shelf-life of in vitro and in vivo biosensors, bioreactors and medical devices containing biologically fragile component(s), as well as making it possible and easy to package, store and transport such devices.

### STATEMENT REGARDING FEDERAL FUNDING

This invention was made with government support under Grant Nos. NS062019 and NS089688 awarded by the National Institutes of Health. The government has certain rights in the invention.

### SUMMARY

In one aspect of the present invention, a method of preparing a storage-stable protein-coated article is provided. The method comprises: linking a protein to silica nanoparticles attached to a substrate to produce a coated article; drying the coated article; and placing the dried, coated article in packaging, wherein none, or no more than 10%, 5%, or 1% of the activity of the protein is lost on storage of the dried, coated article in the packaging for at least 24 hours or at least 7 days at 25° C.

In another aspect, a kit is provided. The kit comprises: a dried protein-coated article, comprising: a substrate; silica nanoparticles linked to the substrate; a protein linked to the silica nanoparticles; and packaging containing the dried protein-coated article, wherein none, or no more than 10%, 5%, or 1% of the activity of the protein is lost on storage of the dried, coated article in the packaging for at least 24 hours or at least 7 days at 25° C.

In yet another aspect, a method of treating a subject is provided. The method comprises implanting into a patient a dried protein-coated article stored in a dry state for at least 24 hours, the article comprising: a substrate; silica nanoparticles linked to the substrate; and a protein linked to the silica nanoparticles, wherein none, or no more than 10%, 5%, or 1% of the activity of the protein is lost on storage of the dried, coated article in packaging for at least 24 hours or 7 days at 25° C.

The following numbered clauses describe exemplary aspects, embodiments, and/or examples of the present invention.

Clause 1. A method of preparing a storage-stable protein-coated article, comprising:
linking a protein to silica nanoparticles attached to a substrate to produce a coated article;
drying the coated article; and
placing the dried, coated article in packaging,
   wherein none, or no more than 10%, 5%, or 1% of the activity of the protein is lost on storage of the dried, coated article in the packaging for at least 24 hours or at least 7 days at 25° C.
   Clause 2. The method of clause 1, wherein the dried, coated article is hermetically-sealed in the packaging.
   Clause 3. The method of clause 1, wherein the silica nanoparticles have an average size ranging from 10 nm to 500 nm.
   Clause 4. The method of clause 1, wherein the silica nanoparticles are thiol-modified.
   Clause 5. The method of any one of clauses 1-4, wherein the silica nanoparticles are prepared by a Stöber process, for example using tetraethyl orthosilicate.
   Clause 6. The method of clause 5, further comprising incorporating a mercapto-silane, such as mercaptopropyl trimethoxysilane, into the silica nanoparticle to produce a thiol-modified silica nanoparticle.
   Clause 7. The method of any one of clauses 4-6, wherein the substrate comprises amine groups and the thiol-modified silica nanoparticles are linked to the substrate by a GMBS ((N-[γ-maleimidobutyryloxy]succinimide ester), including sulfa-GMBS (N-[γ-maleimidobutyryloxy]sulfosuccinimide ester)) cross-linker.
   Clause 8. The method of any one of clauses 4-7, wherein the protein is linked to the thiol-modified silica nanoparticles by a GMBS cross-linker.
   Clause 9. The method of any one of clauses 1-8, further comprising sterilizing the protein-coated article.
   Clause 10. The method of any one of clauses 1-9, wherein the substrate is glass, ceramic, silicon, or plastic.
   Clause 11. The method of any one of clauses 1-9, wherein the substrate is an electrode, such as a silicon electrode, that is optionally activated under oxygen plasma.
   Clause 12. The method of any one of clauses 1-9, wherein the substrate is a cell-contacting surface of a tissue culture vessel.
   Clause 13. The method of any one of clauses 1-12, wherein the protein is a cell adhesion molecule.
   Clause 14. The method of any one of clauses 1-13, wherein the protein is an L1 polypeptide (L1 or a cell-adhesion-promoting fragment thereof).
   Clause 15. The method of any one of clauses 1-12, wherein the protein is an enzyme or a binding reagent, such as an antibody or an antibody fragment.
   Clause 16. The method of any one of clauses 1-15, wherein the coated article is air-dried or lyophilized.
   Clause 17. The method of any one of clauses 1-16, further comprising linking the nanoparticles to the substrate using a suitable crosslinker to covalently link the nanoparticles to the substrate.
   Clause 18. A kit comprising:

a dried protein-coated article, comprising:
a substrate;
silica nanoparticles linked to the substrate;
a protein linked to the silica nanoparticles; and

packaging containing the dried protein-coated article,

wherein none, or no more than 10%, 5%, or 1% of the activity of the protein is lost on storage of the dried, coated article in the packaging for at least 24 hours or at least 7 days at 25° C.
Clause 19. The kit of clause 18, wherein the dried, coated article is hermetically-sealed in the packaging.
Clause 20. The kit of clause 18 or 19, wherein the silica nanoparticles have an average size ranging from 10 nm to 500 nm.
Clause 21. The kit of any one of clauses 18-20, wherein the silica nanoparticles are linked to the protein and/or the substrate via a linker comprising a thioether bond. Clause 22. The kit of clause 21, wherein the silica nanoparticles are linked to the protein and/or the substrate via a linker comprising a GMBS ((N-[γ-maleimidobutyryloxy]succinimide ester) residue.
Clause 23. The kit of any one of clauses 18-22, wherein the article is sterile.
Clause 24. The kit of any one of clauses 18-23, wherein the substrate is glass, ceramic, silicon, or plastic.
Clause 25. The kit of any one of clauses 18-24, wherein the substrate is an electrode, such as a silicon electrode.
Clause 26. The kit of any one of clauses 18-24, wherein the substrate is a cell-contacting surface of a tissue culture vessel.
Clause 27. The kit of any one of clauses 18-26, wherein the protein is a cell adhesion molecule.
Clause 28. The kit of clause 27, wherein the protein is an L1 polypeptide.
Clause 29. The kit of any one of clauses 18-26, wherein the protein is an enzyme or a binding reagent, such as an antibody or an antibody fragment.
Clause 30. The kit of any one of clauses 18-29, wherein the coated article is air-dried or lyophilized.
Clause 31. A method of treating a subject, comprising implanting into a patient a dried protein-coated article stored in a dry state for at least 24 hours, the article comprising:

a substrate;

silica nanoparticles linked to the substrate; and

a protein linked to the silica nanoparticles,

wherein none, or no more than 10%, 5%, or 1% of the activity of the protein is lost on storage of the dried, coated article in packaging for at least 24 hours or 7 days at 25° C.
Clause 32. The method of clause 31, further comprising hydrating the dried protein coated article prior to implanting the article into the patient.
Clause 33. The method of clause 31 or 32, wherein the silica nanoparticles have an average size ranging from 10 nm to 500 nm.
Clause 34. The method of any one of clauses 31-33, wherein the silica nanoparticles are linked to the protein and/or the substrate via a linker comprising a thioether bond.
Clause 35. The method of clause 34, wherein the silica nanoparticles are linked to the protein and/or the substrate via a linker comprising a GMBS (N-[γ-maleimidobutyryloxy]succinimide ester or sulfo-N-[γ-maleimidobutyryloxy]succinimide ester) residue.
Clause 36. The method of any one of clauses 31-35, wherein the substrate is glass, ceramic, silicon, or plastic.
Clause 37. The method of any one of clauses 31-36, wherein the substrate is an electrode, such as a silicon electrode.
Clause 38. The method of any one of clauses 31-37, wherein the substrate is a cell-contacting surface of a tissue culture vessel.
Clause 39. The method of any one of clauses 31-38, wherein the protein is a cell adhesion molecule.
Clause 40. The method of clause 39, wherein the protein is an L1 polypeptide (L1 or a cell-adhesion-promoting fragment thereof).
Clause 41. The method of any one of clauses 31-8, wherein the protein is an enzyme or a binding reagent, such as an antibody or an antibody fragment.

### BRIEF DESCRIPTION OF THE DRAWINGS

FIGS. 1A-1C provide exemplary protein sequences for L1 proteins, also known as L1-CAM or CD171.

FIG. 2. Protein stability at 37° C. L1 was immobilized on smooth glass coverslips, or onto thiol-modified nanoparticle (TNP) modified glass coverslips, and incubated for up to four weeks as shown on the x-axis. Primary neurons were then cultured directly on the protein modified substrates to examine if bioactivity decreased with time. Neurite outgrowth is normalized to control coverslips (0 days, TNP modified). *p<0.05

FIG. 3. Protein Stability After Drying. L1 was immobilized on smooth and TNP modified coverslips then air dried and stored for up to 7 days. Primary neurons were then cultured on the substrates and the neurite outgrowth was measured after 2 days in culture. ***p<0.001. For both smooth and TNP clusters, the data is provided in the following order, left to right, No L1, L1-wet, L1 dried 1 day, and L1 dried 7 days.

FIG. 4. Nanoparticles coat the entire electrode. (A) TNP modification demonstrating the immobilization of particles to the shank and electrode sites (red circles). Impedance measurements were taken prior to nanoparticle immobilization, after immobilization, and after L1 was immobilized to the particles and only a modest increase in impedance was observed (n=16).

FIG. 5. Neurite outgrowth measured on L1 or L1 on TNP after extended incubation periods. Fresh L1 was prepared on the day of culture and serves as a control. L1 modified substrates incubated for 4-weeks have significantly decreased neurite outgrowth, while L1 on TNP maintained its bioactivity (n=27). ****p<0.001

FIG. 6. Representative images of neurons grown on L1 modified substrates after storage overnight at room temperature. The neurite outgrowth was not significantly different between any group (n=12, freeze dry—left bar, air dry—middle bar, wet—right bar).

FIG. 7. Electrophysiological recording metrics. (A) Impedance measurements normalized to pristine electrodes. L1+TNP plateaus at a lower value and maintains its impedance throughout the experiment, while control and L1 modified electrode have a decrease in impedance at later time points. (B) The percentage of electrode sites recording identifiable units. (C) The noise floor of all channels.

FIGS. 8A and 8B. SEM images of nanoparticles immobilized onto parylene C surface. (FIG. 8A (A)) low magnification image showing the nanoparticles binding to the parylene coating. The parylene C has folded into-itself, yet the nanoparticles are still bound (bar=10 μm). (FIG. 8A (B)) Higher magnification image showing the nanoparticle distribution (bar=1 μm). (FIG. 8B (C)) Additional images demonstrating the ability of nanoparticles to repeatably bind to the parylene C surface (bar=1 μm). (FIG. 8B (D)) Primary neurons growing on L1-nanoparticle modified substrates.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with “including”, “containing”, or “characterized by”. The term “consisting essentially of” limits the scope of a claim to the specified materials or steps and those that do not materially affect basic and novel characteristic(s). The term “consisting of” excludes any element, step, or ingredient not specified in the claim. As used herein, embodiments “comprising” one or more stated elements or steps also include, but are not limited to embodiments “consisting essentially of” and “consisting of” these stated elements or steps.

Provided herein are methods and kits relating to a storage-stable dried article having a coating comprising functionally active polypeptides. Polypeptide activity is maintained even when stored dried and later reconstituted, e.g., hydrated. In further detail, the article comprises a surface, and the surface comprises, e.g. is decorated with, silica nanoparticles that are linked (covalently bound via a linker) to the surface. Polypeptides are linked to the silica nanoparticles attached to the surface.

The surface may be any suitable surface for coating with the polypeptide. The surface may be amorphous or crystalline silica or siliceous material such as glass, quartz, fused silica, or fused quartz. The surface may be polymeric, such as a polycarbonate, polyethylene, or acrylic plastic surface. The surface may be a metal or ceramic. In one example, the surface comprises silicon oxide, and in another, metal iridium. The surface may be a vapor-deposited conformal coating, such as Parylene C or N. Parylene is a poly(p-xylylene) polymer, with Parylene C having a pendant Cl on the aromatic ring.

The article may be any item that benefits from having a polypeptide coating. The nanoparticle-modified surface not only allows for dense packing of the polypeptides on the surface, but unexpectedly facilitates essentially complete preservation of polypeptide activity after drying and reconstitution (e.g., hydrating). This preservation of activity is not seen on surfaces that are not coated with nanoparticles, or with polypeptide-coated nanoparticles that are not surface-bound. Examples of devices that would benefit from a polypeptide coating, include, without limitation: implantable or prosthetic devices, such as catheters, shunts, stents, coils, pacemakers, electrodes, sensors, or orthopedic implants; sensors; cell-culture vessels or surfaces thereof, such as, without limitation, plates, multi-well plates, flasks, bioreactor surfaces, culture chambers, or tubes; assay devices, such as ELISA or array devices; body-contacting devices; surfaces with anti-fouling or antimicrobial polypeptide modifications; catalytic surfaces having an active enzyme affixed thereto.

In one example, the article is an electrode, e.g., as described in the Examples, below, comprising a coating of a cell-adhesion molecule, such as an L1 protein, which, upon drying, retains full activity and which, after reconstitution and implantation, produces an electrode with superior electronic properties, when compared to an un-modified or an L1-coated electrode that does not include the silica nanoparticles.

As indicated above, activity of a polypeptide, such as a cell-adhesion molecule or an enzyme, is retained after drying. Activity is retained for at least one week in a dried state stored in suitable packaging at room temperature, e.g., 25° C. Activity may be fully (100%) retained, or is substantially retained to at least 90%, at least 95%, at least 97.5%, or at least 99% of its activity prior to drying. As an example, little or no difference is seen between cell growth, quantitatively and/or qualitatively, on a polypeptide-coated surface that has been previously dried, as compared to a polypeptide-coated surface that has not been previously dried. For sensors that rely on enzymatic activity, such enzymatic activity is retained on a polypeptide-coated surface that has been previously dried, as compared to a polypeptide-coated surface that has not been previously dried. Electrodes may be evaluated based on retained electronic efficacy after implant, such as impedance, yield, noise floor, or signal-to-noise ratio. Polypeptides used in an assay can be evaluated based on retention of functionality in that assay.

The surface comprises, or is modified to provide, reactive groups that react with a linker (crosslinker). Examples of suitable reactive groups include, without limitation, alkene, alkyne, amine, carboxyl, carbonyl, hydroxyl, thiol (sulfhydryl), phosphate, and epoxy or oxirane groups. For example, a silicon or silica surface may be modified with a silane compound, such as a glycidyl-modified silane such as (3-glycidyloxypropyl) trimethoxy silane to provide glycidyl (epoxy) functionality, or an amine-modified silane, such as aminopropyl triethoxysilane (ATS), to provide amine functionality (amine reactive or functional groups). Polymer materials may be functionalized in any useful manner to include reactive groups that can be linked to a nanoparticle. Based on the present disclosure, a person of skill may select suitable reactive groups and crosslinkers for linking nanoparticles both to the surface of the article and to the polypeptide for coating the article.

The density of nanoparticles on the surface may be modified by adjusting the number of reactive groups on the surface. That said, it may be desirable to maximize the number of reactive groups on the surface of the article to more fully cover the surface of the article with the nanoparticles and polypeptide. The surface may be 100% covered with nanoparticles, that is, with the maximum number of nanoparticles that would fit on the surface, considering stacking of the nanoparticles, or optimized packing, such as in a hexagonal packing, such as, for 100 nm nanoparticles, approximately 10⁸ particles per mm², or for 10 nm nanoparticles, approximately 10¹⁰ particles per mm², depending on packing arrangement, size distribution, and regularity of the nanoparticles. Further, reactions between nanoparticles may produce ordered 3-dimensional structures, greatly increasing the number of reactive groups.

Any suitable polypeptide may be linked to the nanoparticles of the article. A “polypeptide” is a chain of at least three amino acid residues, e.g. 10 or more amino acid residues, linked conventionally by an amide bond. Polypeptide as a class includes proteins and oligopeptides, and includes both natural and synthetic polypeptides, such as natural proteins or modified or chimeric versions of natural proteins. A protein may comprise one or more polypeptide chains. As such an article comprising a polypeptide may comprise a protein with a single polypeptide chain or a protein comprising two or more polypeptide chains. An enzyme is a catalytically-active protein. Cell adhesion molecules (CAMs) mediate cell-to-cell and/or cell-to-ECM (extracellular matrix) interactions, and by doing so they may trigger intracellular responses affecting intracellular signaling, cytoskeletal organization and/or gene expression. Major so called ‘superfamilies’ of cell adhesion molecules have been characterized, including IgCAMs, cadherins, selectins, and integrins. L1 Cell Adhesion Molecule (L1 CAM or L1), Neural Cell Adhesion Molecule (NCAM or, e.g., CD56), Membrane Glycoprotein MRC OX-2 (MOX-2, e.g., CD200), Integrin-Associated Protein (IAP, e.g., CD47), or Chemokine, CX3C Motif, Ligand 1 (CX3CL1, or fractalkine) are non-limiting examples of CAMs that may be bound to surface-bound nanoparticles as described herein. CAMs may also include oligopeptides, such as RGD or RGD-containing polypeptides, or similar CAM motifs. Polypeptides or proteins useful in the articles and methods described herein include, for example and without limitation, enzymes for catalysis and sensing, e.g., biosensing, antimicrobial, antibody or antigen for immunoassays, and anticoagulant proteins or polypeptides.

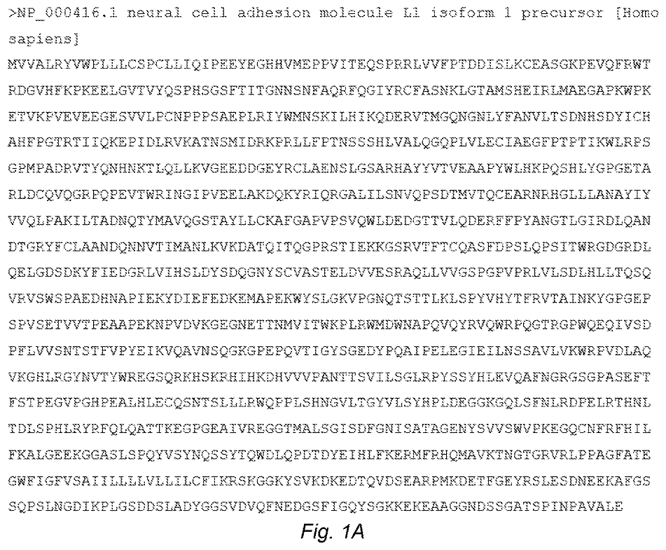

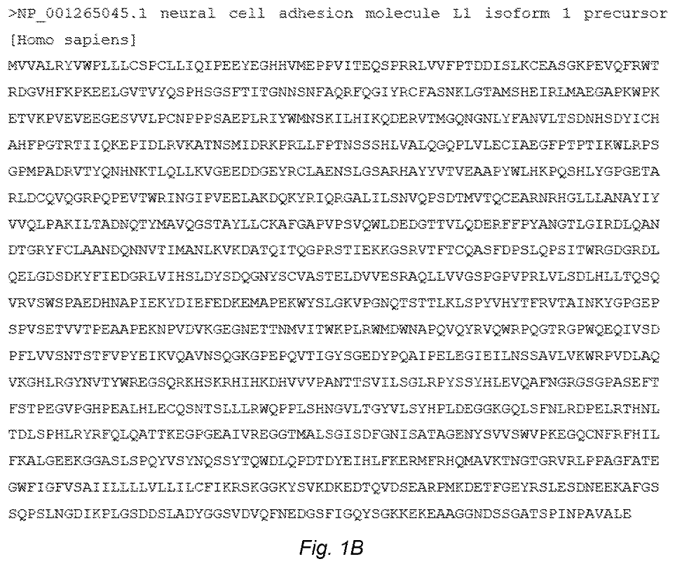

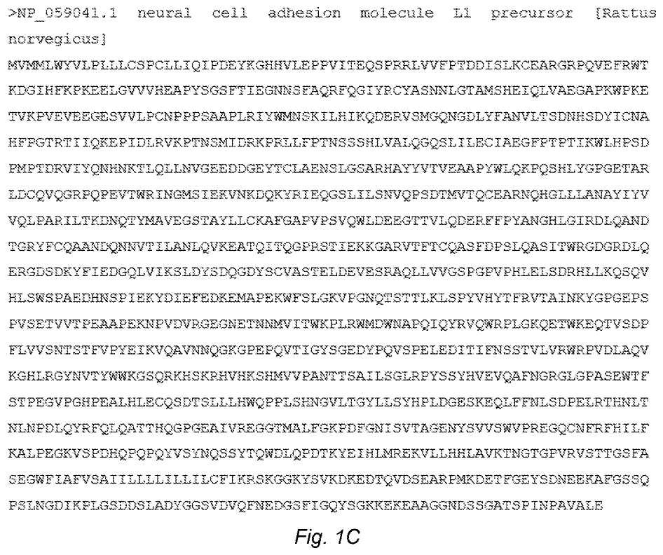

L1 Cell Adhesion Molecule (L1, L1CAM, or CD171 (human form, e.g., HGNC: 6470 Entrez Gene: 3897 Ensembl: ENSG00000198910 OMIM: 308840 UniProtKB: P32004) is an axonal glycoprotein belonging to the immunoglobulin supergene family. The ectodomain, consisting of several immunoglobulin-like domains and fibronectin-like repeats (type III), is linked via a single transmembrane sequence to a conserved cytoplasmic domain. This cell adhesion molecule plays an important role in nervous system development, including neuronal migration and differentiation. Multiple human isoforms of L1 have been identified, examples of which include, without limitation: NM_000425.5 and NP_000416.1 (FIG. 1A), neural cell adhesion molecule L1 isoform 1 precursor and NM_001278116.2 and NP_001265045.1 (FIG. 1B), neural cell adhesion molecule L1 isoform 1 precursor. An exemplary rat isoform is described in NM_017345.1 and NP_059041.1 (FIG. 1C) neural cell adhesion molecule L1 precursor. mRNA sequences are broadly-known, and can be codon-optimized for expression. Also, portions, isoforms, sequence variants, alleles, and cross-reactive interspecies variants forms may be used effectively in the devices and methods described herein. Other cell-adhesion molecules may be linked to a surface of an article described herein. L1 proteins or L1 polypeptides, along with any other protein, may be isolated from tissue or prepared by any useful recombinant method, and therefore may include mature, processed versions of the protein, which may include carbohydrate-modified versions thereof, or functional portions or fragments thereof.

The polypeptide linked to the nanoparticles may be a “binding reagent”, which is a reagent, compound or composition, e.g., a ligand, able to specifically bind a target compound. Binding reagents include, without limitation, antibodies (polyclonal, monoclonal, humanized, etc.), antibody fragments (e.g., a recombinant scFv), antibody mimetics such as affibodies, affilins, affimers, affitins, alphabodies, anticalins, avimers, DARPins, fynomers, monobodies, engineered proteins, antigens, epitopes, haptens, or any target-specific polypeptide or protein binding reagent. In aspects, binding reagents includes as a class: monoclonal antibodies, or derivatives or analogs thereof, including without limitation: Fv fragments, single chain Fv (scFv) fragments, Fab′ fragments, F(ab′)₂ fragments, single domain antibodies, camelized antibodies and antibody fragments, humanized antibodies and antibody fragments, multivalent versions of the foregoing, and any paratope-containing compound or composition; multivalent activators including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; or receptor molecules which naturally interact with a desired target molecule. Innumerable protein binding reagents, such as polyclonal and monoclonal antibodies, and fragments thereof, are available commercially.

An “amino acid” is a compound that has the structure H₂N—C(R)—C(O)OH, where R is a side chain or H, such as an amino acid side chain. An “amino acid residue” represents the remainder of an amino acid when incorporated into a chain of amino acids, such as when incorporated into a recognition reagent as discloses herein, e.g., having the structures —NH—C(R)—C(O)—, H₂N—C(R)—C(O)— (when at the N-terminus of a polypeptide), or —NH—C(R)—C(O)OH (when at the C-terminus of a polypeptide).

A “moiety” is a part of a molecule, and can include as a class “residues”, which are the portion of a compound or monomer that remains in a larger molecule, such as a polymer chain, after incorporation of that compound or monomer into the larger molecule, such as a nucleotide as-incorporated into a nucleic acid or an amino acid as-incorporated into a polypeptide or protein.

The term “polymer composition” is a composition comprising one or more polymers. As a class, “polymers” can include, without limitation, homopolymers, heteropolymers, co-polymers, block polymers, block co-polymers and can be both natural and synthetic. Homopolymers contain one type of building block, or monomer, whereas co-polymers contain more than one type of monomer. An “oligomer” can be a polymer that comprises a small number of monomers, such as, for example, from 3 to 100 monomer residues. As such, the term “polymer” can include oligomers.

A polymer “comprises” or is “derived from” a stated monomer if that monomer is incorporated into the polymer. Thus, the incorporated monomer that the polymer comprises is not the same as the monomer prior to incorporation into a polymer, in that at the very least, certain linking groups are incorporated into the polymer backbone or certain groups are removed in the polymerization process. A polymer is said to comprise a specific type of linkage if that linkage is present in the polymer. An incorporated monomer can be a “residue”. A monomer for a protein or polypeptide is an amino acid.

“Non-reactive”, in the context of a chemical constituent, such as a molecule, compound, composition, group, moiety, ion, etc. can mean that the constituent does not react with other chemical constituents in its intended use to any substantial extent. The non-reactive constituent is selected to not interfere, or to interfere insignificantly, with the intended use of the constituent, moiety, or group in the articles described herein.

As used herein, “alkyl” refers to straight, branched chain, or cyclic hydrocarbon groups including, for example, from 1 to about 20 carbon atoms, for example and without limitation C₁₋₃, C₁₋₆, C₁₋₁₀ groups, for example and without limitation, straight, branched chain alkyl groups such as methyl, ethyl, propyl, butyl, pentyl, hexyl, heptyl, octyl, nonyl, decyl, undecyl, dodecyl, and the like. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of straight alkyl groups include methyl, ethyl, propyl, butyl, pentyl, hexyl, heptyl, octyl, nonyl, and decyl. Branched alkyl groups comprises any straight alkyl group substituted with any number of alkyl groups. Non-limiting examples of branched alkyl groups include isopropyl, isobutyl, sec-butyl, and t-butyl. Non-limiting examples of cyclic alkyl groups include cyclopropyl, cyclobutyl, cyclopentyl, cyclohexyl, cycloheptlyl, and cyclooctyl groups. Cyclic alkyl groups also comprise fused-, bridged-, and spiro-bicycles and higher fused-, bridged-, and spiro-systems. A cyclic alkyl group can be substituted with any number of straight, branched, or cyclic alkyl groups. “Substituted alkyl” can include alkyl substituted at 1 or more (e.g., 1, 2, 3, 4, 5, or even 6) positions, which substituents are attached at any available atom to produce a stable compound, with substitution as described herein. “Optionally substituted alkyl” refers to alkyl or substituted alkyl. “Halogen,” “halide,” and “halo” refers to —F, —Cl, —Br, and/or —I. “Alkylene” and “substituted alkylene” can include divalent alkyl and divalent substituted alkyl, respectively, including, without limitation, methylene, ethylene, trimethylene, tetramethylene, pentamethylene, hexamethylene, hepamethylene, octamethylene, nona methylene, or decamethylene. “Optionally substituted alkylene” can include alkylene or substituted alkylene.

A silica nanoparticle is a particle having a size ranging from, for example, 1 nm to 500 nm. Silica nanoparticles are formed from a network of silicate moieties generally comprising four oxygen atoms surrounding a central silica atom, e.g. in a tetrahedral configuration, with silicon and oxygen atoms alternating. Size of the nanoparticles may be determined by any useful method, including, for example and without limitation, TEM imaging, interactive force apparatus (IFA), electrical mobility analysis using a differential mobility analyzer (DMA), dynamic light scattering (DLS), centrifugal liquid sedimentation (CLS), small-angle X-ray scattering (SAXS), and particle tracking analysis (PTA), among other methods. Silica nanoparticles may be prepared by any method, such as a sol-gel method, such as the Stöber process in which, for example, tetraethyl orthosilicate (Si(OCH₂CH₃)₄, also Si(OEt)₄ or TEOS) is hydrolyzed by an alcohol in the presence of ammonia, and condenses to form silica nanoparticles of uniform and controllable size. Thiol-modified silica nanoparticles may be prepared using a Stöber process performed with an orthosilicate in the presence of a mercapto-alkyloxysilane. The orthosilicate may be an tetraalkyl, e.g., C₁-C₆ alkyl, orthosilicate, such as tetraethyl orthosilicate (TEOS). The mercapto-alkyloxysilane may be a mercaptoalkyl trialkyloxysilane, such as a mercapto-(C₁-C₆ alkyl) tri(C₁-C₆ alkyl)oxysilane, for example and without limitation mercaptopropyl trimethoxysilane (MTS). Similar methods and compounds may be used to prepare functionalized nanoparticles with other reactive groups that may be used to link the nanoparticles to a surface and to a polypeptide, including, without limitation, carboxyl, amine, phosphate, hydroxyl, glycidyl, etc.

A reactive group is a group or moiety of a molecule that can be reacted with another reactive group of another molecule to link the two molecules, and can include, without limitation, carboxyl, amine, phosphate, hydroxyl, glycidyl, N-hydroxysuccinimide esters, maleimides, azides, tetrazine, alkynes, hydrazides, cycloalkyne, among others. Click chemistry pairs, such as strained cyclooctenes and tetrazines, may be used to join elements of the articles described herein.

A “linker” (aka, crosslinker) is a compound or a moiety that covalently attaches two molecules, e.g. the surface, nanoparticles, and polypeptide described herein. The linker moiety incorporated into a larger structure may be a non-reactive moiety, and, in some aspects includes from 5-25 carbon atoms (C₁-C₁₀), optionally substituted with a hetero-atom, such as a N, S, or O, or a non-reactive linkage, such as an amide linkage (peptide bond) formed by reacting an amine with a carboxyl group. Examples of C₁-C₁₀ alkylenes include linear or branched, alkylene (bivalent) moieties such as a methylene, ethylene, trimethylene, tetramethylene, pentamethylene, hexamethylene, hepamethylene, octamethylene, nonamethylene, or decamethylene moiety (e.g., —CH₂—[CH₂]ₙ—, where n=1 to 9). The linkers may comprise ethylene oxide (e.g., —O—CH₂—CH₂— or —CH₂—CH₂—O—) moieties.

The linker or linking group may be an organic moiety that connects two parts of the article described herein, e.g., covalently attaches the surface to the nanoparticles or the nanoparticles to the polypeptide. Linkers typically comprise a direct bond or an atom such as oxygen, nitrogen, phosphorus, or sulfur, a unit such as, C(O), C(O)NH, SO, SO₂, SO₂NH, or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, in which one or more carbons, e.g., methylenes or methylidynes (—CH═) is optionally interrupted or terminated by a hetero atom, such as O, S, or N, substituted or unsubstituted aryl, substituted or unsubstituted heteroaryl, substituted or unsubstituted heterocyclic. In one aspect, the linker may comprise or consist of between about 5 to 25 atoms, e.g., 5-20, 5-10, e.g., 5, 6, 7, 8, 9, 10, 11, 12, 13, 14, 15, 16, 17, 18, 19, or 20 atoms, or a total of from 1 to 10, e.g., 1, 2, 3, 4, 5, 6, 7, 8, 9, or 10 C and heteroatoms, e.g., O, P, N, or S atoms. The linker may have a molecular weight, based on the atomic mass of its constituent atoms, of less than 500 Daltons (Da) or less than 400 Da.

For linkage of a thiol to an amine, such as for linking thiol-modified nanoparticles to an amine-functionalized substrate and/or an amine group of a protein, a crosslinking compound such as N-maleimidobutyryl-oxysuccinimide ester (GMBS), or an N-maleimidoalkanoic-oxysuccinimide ester where the alkanoic moiety may be C₂₋₆ alkanoic (having the structure —[CH₂]ₙ—C(O)O—, where n is 1-5), such as, without limitation a 3-(maleimido)propionic acid N-hydroxysuccinimide ester (BMPA), a 3-maleimidocaproic acid N-hydroxysuccinimide ester (EMCS, also 6-maleimidohexanoic acid N-hydroxysuccinimide ester), or an N-α-maleimidoacet-oxysuccinimide ester (AMAS), may be employed, resulting in the residue or linker moiety:

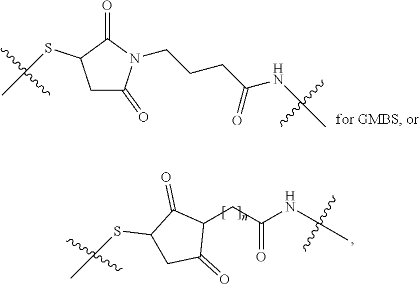

where n is 1-5,
where linkage of the amine group to, e.g., GMBS results in formation of an amide bond, and linkage of the thiol group to GMBS results in formation of a thioether bond. That is, the residue comprises an alkyl moiety with an amide bond at one end and a maleimide thioether moiety at the other. In the context of the present disclosure linkage of thioether-modified silica nanoparticles to amine-modified surfaces and proteins, is a simple, straightforward process, and may be preferred. That said, many crosslinking reagents and methods are known to those of ordinary skill (see, e.g., Thermo Scientific Crosslinking Technical Handbook, 2012, Thermo Fisher Scientific, Inc., describing different methods of linking molecules). Reference to a crosslinker, such as an N-maleimidoalkanoic-oxysuccinimide ester, such as GMBS, includes suitable salt forms thereof to impart water-solubility to the crosslinker sulfo-salts, for example sulfo-N-maleimidoalkanoic-oxysuccinimide ester, such as sulfo-GMBS.

The dried, polypeptide-coated article described herein may be provided in a kit. A kit comprises the dried article within packaging. The packaging may be a vial, bag, Mylar or foil pouch, a box, or any other shape suitable for containing or configured to contain the article or multiple articles. The packaging may be hermetically sealed, preventing or substantially preventing fluid or gas exchange. The article may be packaged in the kit under sterile conditions and/or the kit may be sterilized by any useful, non-destructive method. None, or no more than 10%, 5%, or 1% of the activity of the protein is lost on storage of the dried, coated article in packaging for at least 24 hours or 7 days at 25° C. The packaging may be suitable for the storage, transport, and/or commercial distribution of the dried article.

Also provided herein is a method of preparing a storage-stable protein-coated article. The method comprises linking a polypeptide, e.g. a protein, to silica nanoparticles attached to a substrate to produce a coated article. The article is then dried and is placed in packaging suitable for the storage, transport, and/or commercial distribution of the dried article and may be hermetically sealed in the packaging. None, or no more than 10%, 5%, or 1% of the activity of the protein is lost on storage of the dried, coated article in the packaging for at least 24 hours or at least 7 days at 25° C. The method also may include linking the nanoparticles to the substrate using a suitable crosslinker to covalently link the nanoparticles to the substrate.

By “sterilized” it is meant acceptably and practicably sterile for the end use of the article, for example, in reference to commercial and/or regulatory requirements. Articles described herein may be sterilized by any useful method, so long as it does not interfere substantially and practicably with the end-use of the article.

“Dried” or “drying” refers to removal of aqueous liquid from an article, and may be accomplished by any suitable method, such as by aspiration and air drying, vacuum-drying, or lyophilization or freeze-drying drying methods.

Also provided herein are methods of treating a patient. The method comprises hydrating a dried protein-coated article stored in a dry state for at least 24 hours, the article comprising: a substrate; silica nanoparticles linked to the substrate; and a polypeptide, e.g. a protein, linked to the silica nanoparticles, wherein none, or no more than 10%, 5%, or 1% of the activity of the polypeptide is lost on storage of the dried, coated article in packaging for at least 24 hours or 7 days at 25° C.; and implanting the hydrated article into the patient. The article is, for example and without limitation, an implantable or prosthetic device, such as a catheter, a shunt, a stent, a coil, a pacemaker, an electrode, a sensor (e.g., a biosensor), a drug-delivery device, or an orthopedic implant. A biosensor may have a surface linked to silica nanoparticles, and comprising at least two portions, with nanoparticles of the first portion are linked to a first polypeptide or protein, such as a CAM, and nanoparticles of the second, sensor portion are linked to an enzyme that produces a detectable signal in the presence of an analyte, such as a calorimetric or fluorescent signal, or an electrical signal, for example and without limitation, as generated by an electrochemical, e.g. a redox reaction.

Biosensors may include a biological recognition element, a transducer, and a signal processing system. For enzyme-based biosensors, the enzyme is the recognition element, and is immobilized as described herein on the transducer surface in order to maintain enzyme activity. The enzyme-based biosensor detects the presence of certain analytes by measuring any detectable change, such as, without limitation: proton concentration (H⁺), the release or uptake of gases such as CO₂, NH₃, or O₂), light emission, absorption or reflectance, or heat emission. These changes take place during substrate consumption or product formation of an enzymatic reaction. The transducer converts those changes into detectable signals (electrical, optical, or thermal) that are used to identify analytes of interest. Transducer types include, without limitation: electrochemical, optical, thermal/calorimetric, and piezoelectric biosensors. Non-limiting examples of enzymes, which may be combined, or used individually, useful in biosensors include: urease, creatinine deaminase, l-asparaginase, glucose oxidase, β-galactosidase, peroxidase, D-amino acid oxidase, acetylcholinesterase, butyrylcholinesterase, uricase, alcohol oxidase, tyrosinase, lactate dehydrogenase and pyruvate oxidase, glycerol catalase, yeast hexokinase, bacterial luciferase co-immobilized with other NAD(P)H-dependent enzymes such as sorbitol dehydrogenase, alcohol dehydrogenase, and malate dehydrogenase, laccase, microperoxidase-11, NADH dehydrogenase, β-lactamase, catalase, hexokinase, cholesterol oxidase, trypsin, antibodies, horseradish peroxidase, cholinesterase, cholesterol esterase, glutamate oxidase, γ-aminobutyrate aminotransferase, succinic semialdehyde dehydrogenase, superoxide dismuatase (See, e.g., Nguyen, Hoang Hiep et al. “Immobilized Enzymes in Biosensor Applications.” Materials (Basel, Switzerland) vol. 12,1 121. 2 Jan. 2019, doi:10.3390/ma12010121).

As used herein, a “patient” or “subject” may be an animal, such as a mammal, including, but not limited to, a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose).

As used herein, the terms “treating”, or “treatment” can refer to a beneficial or specific result, such as improving one of more functions, or symptoms of a disease, or can facilitate monitoring a disease status, such as through implantation of an electrode for monitoring nerve/neuronal signals. “Treatment” can also mean prolonging survival as compared to expected survival in the absence of treatment.

### Example 1—Stability and Function of Nanoparticle-Immobilized Protein on Silicon and Glass Substrates

Thiol-modified silica nanoparticles are capable of being immobilized on silicon and glass substrates, producing a highly textured surface which greatly increasing the protein binding density. Surface modification with nanoparticles also improve the protein stability in both physiological conditions and ambient dry condition.

Silica nanoparticles are used as a textural surface modification for increasing the bioactive lifetime of immobilized proteins, while also enabling the drying and room-temperature storage of protein modified substrates. Thiol-modified silica nanoparticles were immobilized to glass substrates, creating a highly textured surface. The protein studied was the neural adhesion molecule L1-CAM, a protein which has shown to modulate the inflammation of central nervous tissue in response to implantation of microelectrodes. L1 immobilized to the textured substrate was capable of maintaining its activity for 4 weeks when stored in buffered saline at 37° C. Further, the L1 modified substrates could be air dried and stored at room temperature under ambient conditions for at least one week. Together, these results demonstrate that a nanoparticle textural modification is sufficient to both greatly extend the lifetime of immobilized proteins, and to enable off-site immobilization and shipping of bioactive surfaces.

### Results and Discussion

To improve the stability of protein surface modifications, we have developed a novel nanoparticle-textured substrate. Silica nanoparticles were chosen due to their biocompatibility and ease of functionalization, enabling creation of an immobilizable thiol-modified silica nanoparticle (TNP). To bind TNP, a glass coverslip was first pre-treated with an amino-silane to present amine groups on the surface. The thiol-modified nanoparticles were bound to the treated glass coverslips via sulfo-N-maleimidobutyryl-oxysuccinimide ester (GMBS). A second treatment of GMBS enabled the binding of the TNP-texturized slide to the L1 protein.

### In Vitro Stability

The biological lifetime of proteins is limited by their tendency to denature, causing an incorrectly folded tertiary structure and loss of function. To determine if TNP texturized glass was capable of preserving the bioactivity of immobilized L1, modified coverslips were incubated in buffered saline for up to four weeks prior to the introduction of primary neurons. L1 has previously shown to encourage neurite outgrowth when immobilized on the substrate. A smooth control was included to demonstrate the fragility of the bound protein. Smooth L1-modified coverslips were prepared to match the surface chemistry of the TNP-modified coverslips. The glass was first silanized with mercaptopropyl trimethoxysilane, then the L1 was immobilized though the same GMBS chemistry as the TNP-modified glass. With the exception of texture, the smooth and TNP modified coverslips were treated identically.

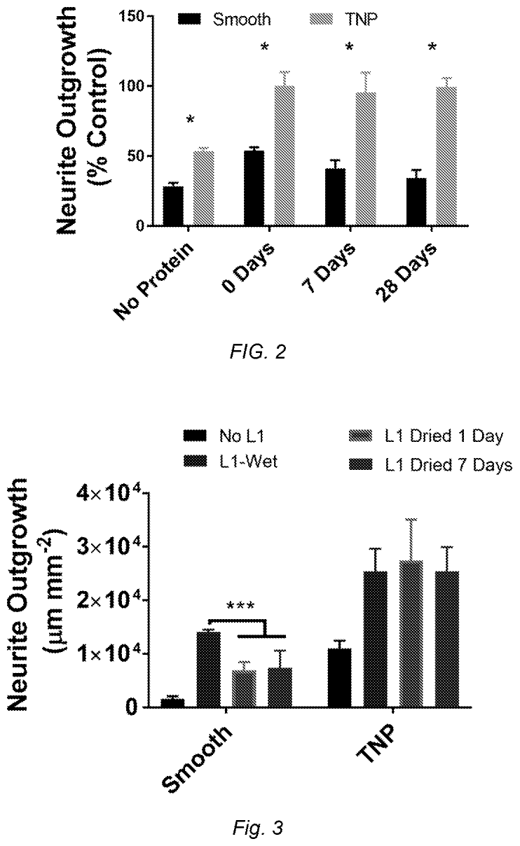

After two days of culture, a significant decrease in the neurite outgrowth was not observed when primary neurons were cultured on L1-TNP substrates (FIG. 2). Conversely, longer incubation times led to a decrease in the neurite outgrowth of cell cultured on smooth-L1 coverslips. Together, these results demonstrate that the L1 protein directly bound to the smooth substrate is susceptible to bioactivity loss, but the additional stability imparted by the TNP-modified substrate is sufficient to maintain the L1 at physiologic conditions for up to four weeks.

### Stability Upon Drying and in Dry Condition

Immobilization of proteins can allow for inert materials to gain impressive biological properties, including enzyme-driven catalysis and bio-integration. However, the difficulty in maintaining the activity of immobilized proteins outside of low temperature-wet conditions makes off-site manufacturing and transportation of protein-modified substrates a challenge. To create a dry-stable protein modified substrate, the same TNP-modified glass and smooth control described above was utilized. L1-modified substrates were dried via aspiration and stored at room temperature (e.g., 25° C.±5° C.) in sterile conditions for up to one week. After storage, primary neurons were cultured directly on the substrate to assess the activity of L1 (FIG. 3). The smooth coverslips lost the majority of their respective neurite outgrowth after drying, while the TNP modified coverslips had no decrease in outgrowth. This change in outgrowth is attributed to the particle modification stabilizing the L1 either by providing a suitable scaffolding structure or by providing an appropriate microenvironment around the protein. These results are highly promising, providing evidence that TNP-L1 modified substrates can be stored and shipped on demand, without need for immediate on-site immobilization.

### Example 2

### Methods:

Nanoparticle Fabrication: 35 ml Di-water, 5 ml triethanolamine, and 8 ml ethanol were added to a round bottom flask and heated to 60° C. while stirring. After 30 minutes, 3 ml tetraethyl orthosilicate (TEOS) was added dropwise over 5 minutes. The reaction progressed for 5-10 minutes, at which point 1 ml of mercaptopropyl trimethoxysilane (MTS) was added. The reaction proceeded for 1 hour, followed by the addition of 250 μl of MTS, and then allowed to react for an additional hour. The nanoparticles were collected by centrifuge and washed with ethanol and Di-water. The resulting silica nanoparticles are coated with active thiol groups from the MTS, henceforth referred to as thiolated nanoparticles (TNP)

In Vitro Preparation: Substrates (silicon, glass) were cleaned in isopropanol by sonicating for 5 minutes. Clean substrates were activated under oxygen plasma for 5 minutes. Activated substrates were silanized by submerging in 2.5 v % silane monomer in anhydrous ethanol for 1 hour at R.T. Silane monomers include MTS, and aminopropyl triethoxysilane (ATS).

Nanoparticle immobilization was performed on ATS modified substrates. ATS modified substrates were washed under ethanol and water, following which the substrates were submerged in GMBS solution, 2 mg ml⁻¹ in phosphate buffered saline (PBS), at 37° C. for 30 minutes. Substrates were washed with water, then submerged in TNP suspension, −10 mg ml⁻¹ in 0.1×PBS, at 37° C. for 1 hour. Substrates were agitated during nanoparticle deposition by placing the reaction well on a heated orbital shaker to maintain temperature. Substrates were collected and washed thoroughly with water, resulting in TNP modified substrates.

MTS and TNP modified substrates were then submerged in GMBS solution, 2 mg ml⁻¹ in phosphate buffered saline, at 37° C. for 30 minutes. Substrates were washed, following which L1 solution, ˜20 μg ml⁻¹ was added to samples and reacted for 30 minutes at 37° C. The resulting substrates were referred to as L1 modified and L1-TNP modified.

In Vivo Preparation: Silicon electrodes were purchased from NeuroNexus. Electrodes were first cleaned in isopropanol, followed by activation under oxygen plasma for 5 minutes. All remaining steps took place in a sterile cell culture hood. The electrode was grabbed with a stereotaxic manipulator by the connecter tab and dipped into a 2.5 v % ATS/ethanol solution for 1 hour at R.T. Care was taken such that only the shank of the electrode was in solution. The electrode was the washed with sterile alcohol and water, then dipped into GMBS solution, 2 mg ml⁻¹ in phosphate buffered saline, at 37° C. for 30 minutes. Substrates were washed with sterile water, then submerged in thiolated nanoparticle suspension, −10 mg ml⁻¹ in 0.1×PBS, at 37° C. for 1 hour. The electrode was agitated during nanoparticle deposition by placing the reaction well on a heated magnetic stir plate, with a small magnetic stir bar in the reaction well. Care was taken to ensure that the shank of the electrode was high above the stir bar, minimizing risk of damage to the device. The probe was then removed, washed with sterile water, and dipped into GMBS solution, 2 mg ml¹ in phosphate buffered saline, at 37° C. for 30 minutes. Finally, the probe was washed and dipped into L1 solution, ˜20 μg ml⁻¹ and reacted for 30 minutes at 37° C.

Surgery: Animals were anesthetized with isoflurane. Once anesthesia was confirmed, the scalp was sterilized with iodine solution, then an incision made to expose the skull. 1 hole was placed on the left hemisphere, 1.5 mm from the midline and 1 mm anterior from lambda, to expose the visual cortex. 3 bone screws were placed in the skull, 2 anterior to bregma on either side of the midline, and 1 on the right hemisphere, mirroring the hole exposing the visual cortex. The modified electrode was then inserted 1.6 mm, such that each electrode site was in the cortex. The hole was sealed with surgical silicone epoxy. The electrode ground and reference wires were wrapped around separate bone screws. Finally, a skull cap of UV-curable dental cement was built on the skull to stabilize the implanted electrode.

Recording of neural activity and electrode impedance was performed on the day of surgery and weekly following the operation. Mice were anesthetized under 0.8-1.0% isoflurane to inhibit motion while maintaining the ability to respond to visual stimuli. All recordings took place in a faraday cage, and a MATLAB script provided the visual stimulus to a 24″ monitor placed outside the cage. Impedance measurements were performed with AutoLab potentiostat. Impedance was sampled between 10 Hz and 32 kHz. The neural data stream was collected by a TDT recording system (RX7, Tucker-Davis Technologies), sampled at 24 kHz. Raw data was filtered between 0.3-5 kHz to isolate neuron spiking events. Single units were identified by a defined threshold of 3.5 standard deviations of the data. Offline spike sorting was performed through a custom MATLAB script SU signal-to-noise ratio (SNR) was calculated from verified units as peak-to-peak amplitude of the mean waveform divided by the noise floor. Channels with SNR between 2 and 3 were manually selected by examining the combination of waveform shape, auto-correlogram, peak threshold crossing offset, and peri-stimulus time histogram (PSTH) with 50 ms bins and candidate units with SNR greater than 3 were manually confirmed by looking at the waveform shape. The noise floor was defined as 2 standard deviations of the filtered data after removal of the single units.

### Results:

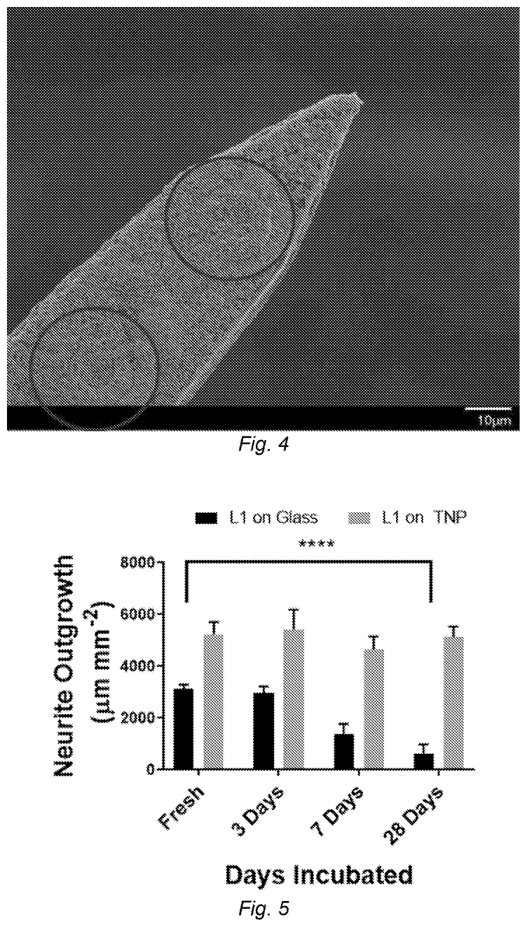

Immobilization of nanoparticles occurred uniformly at both the silicon dioxide surface and the metal electrode sites (FIG. 4). Binding of the nanoparticles to the electrode site was attributed to metal-thiol bonds. Due to the presence of the nanoparticles on the electrode site, a negligible increase in electrode impedance was observed. This increase (<20 kΩ) was not expected to significantly impact the recording/stimulation functionality of the device.

The stability of L1 under physiological conditions was evaluated by incubating the protein modified substrates at 37° C. for up to 4 weeks (FIG. 5). Following incubation, primary cortical neurons were cultured for 36 hours, at which point the cells were fixed in 4% paraformaldehyde (PFA) and stained for β(III)-tubulin. The cells were imaged and neurite process extension was quantified. L1 bioactivity was expected to be directly proportional to process extension. The process extension of primary neurons was preserved on samples for 4 weeks when L1 was immobilized to TNP. However, incubation of substrates where L1 was directly coupled to the glass surface resulted in significantly decreased bioactivity (p<0.001).

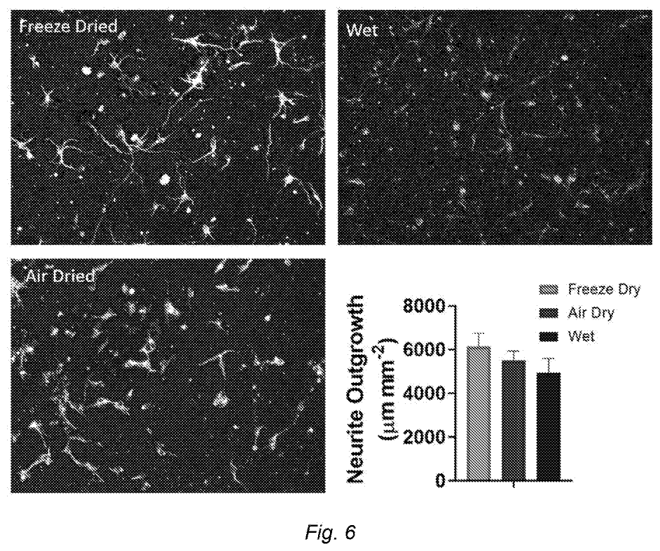

Next, we examined the compatibility of L1-TNP modified substrates with drying and storage (FIG. 6). L1-TNP substrates were created the day prior to culture. The substrates were then divided into three groups, examining the effects of drying the protein under ambient conditions with an aspirator (air drying), freezing and lyophilizing (freeze dried), or letting rest at room temperature overnight in PBS (wet). As with the stability validation, primary neurons were cultured on the samples, and after 36 hour the cells were fixed in 4% PFA and stained. Neurite extension was not significantly different between any groups, indicating that air drying and freeze drying are both viable options to dry and store L1-TNP modified substrates.

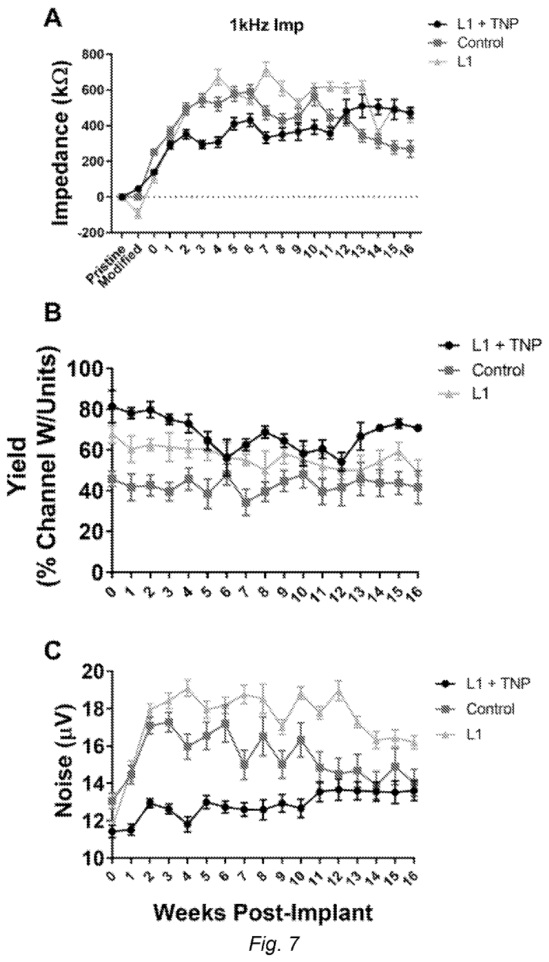

An in vivo experiment was performed to assess the ability of L1-TNP modified electrodes to maintain stable neural recording. Three conditions were examined: Control silicon electrodes, L1 modified electrodes, and L1-TNP modified electrodes. Electrodes were implanted into a mouse model and key electrophysiological parameters such as impedance, yield, and noise floor were examined (FIG. 7). Impedance was normalized to the pristine condition. L1 modified electrode showed a small decrease in impedance following functionalization, likely due to further cleaning of the electrode site during the protein immobilization reaction. L1-TNP modified electrodes plateau at the lowest impedance, approximately 400 kΩ above the pristine value. At longer time periods, the control condition drops in impedance below the L1-TNP modified electrodes, likely due to material failure of the insulation on the control electrode. Yield, defined as the % of electrode channels detecting single unit activity (neuronal spikes), is one of the most important measures of neural electrode recording quality. High yields provide more useful information. L1-TNP electrodes provided the highest yield and the lowest noise floor.

### Example 3—Immobilization of TNP onto Parylene-C Substrates

Methods: Parylene-C was deposited onto glass substrates at a thickness of 3 μm. Parylene substrates were then washed in isopropanol under sonication, then activated with oxygen plasma for 5 minutes. The activated parylene was silanized with 2.5% aminopropyl triethoxysilane in absolute ethanol for 1 hour then washed with absolute ethanol and water. The amine modified parylene was activated with gamma maleimidobutyryl-oxysuccinimide ester (gmbs) by submerging in gmbs solution (2 mg ml−1 in PBS) for 30 minutes. Thiol nanoparticles were immobilized to the substrate by submerging GMBS modified substrates in a nanoparticle suspension, 10 mg ml⁻¹ in 0.1×PBS, for 1 hour at 37° C. The substrates were again washed, then re-submerged in GMBS solution for 30 minutes. Finally, the substrates were washed and submerged in L1 solution, 20 μg ml⁻¹ in PBS, for 1 hour.

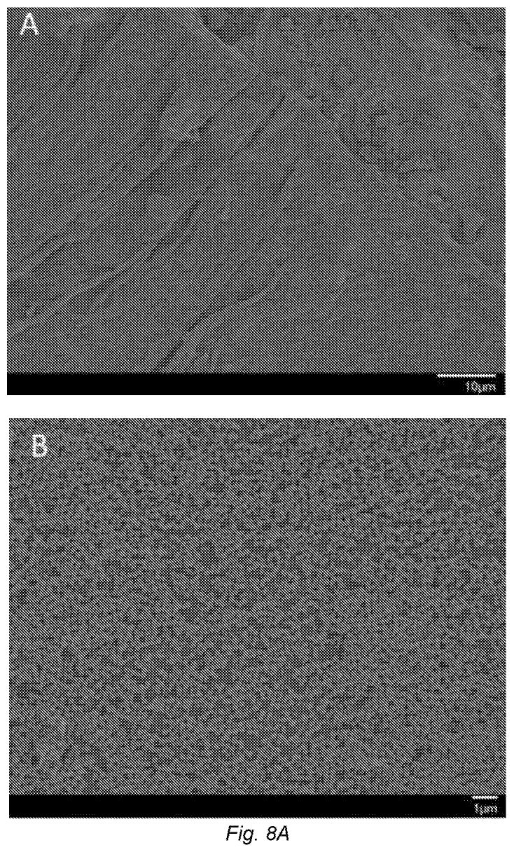

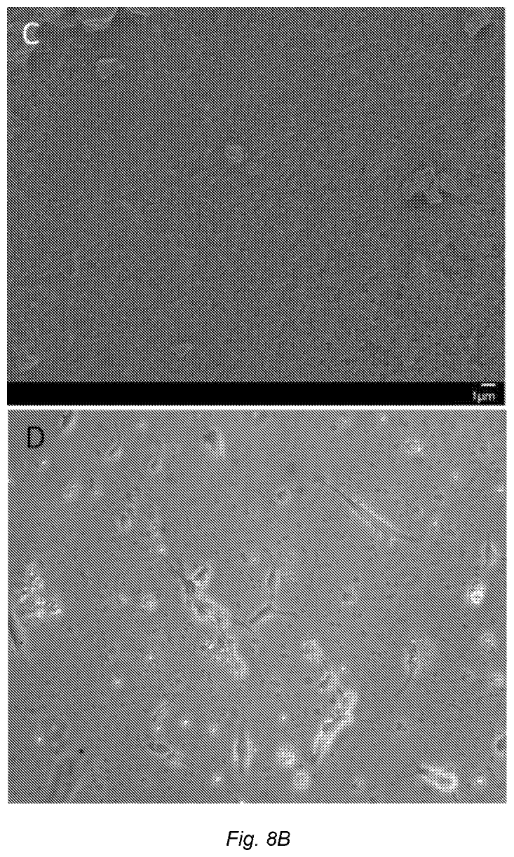

Results: Nanoparticle immobilization was verified under scanning electron microscopy (SEM) (FIGS. 8A and 8B). The immobilized nanoparticles should be functionally identical to the nanoparticles immobilized on glass and silicon substrates. Primary neurons were cultured on L1-TNP modified substrates to demonstrate bioactivity.

Whereas particular embodiments of this invention have been described above for purposes of illustration, it will be evident to those skilled in the art that numerous variations of the details of the present invention may be made without departing from the invention as defined in the appended claims.

1. A method of preparing a storage-stable protein-coated article, comprising:
linking a protein to silica nanoparticles attached to a substrate to produce a coated article;
drying the coated article; and
placing the dried, coated article in packaging,
wherein none, or no more than 10%, 5%, or 1% of the activity of the protein is lost on storage of the dried, coated article in the packaging for at least 24 hours or at least 7 days at 25° C.

2. The method of claim 1, wherein the dried, coated article is hermetically-sealed in the packaging.

3. The method of claim 1, wherein the silica nanoparticles have an average size ranging from 10 nm to 500 nm.

4. The method of claim 1, wherein the silica nanoparticles are thiol-modified.

5. The method of claim 4, wherein the substrate comprises amine groups and the thiol-modified silica nanoparticles are linked to the substrate by a GMBS ((N-[γ-maleimidobutyryloxy]succinimide ester), including sulfo-GMBS (N-[γ-maleimidobutyryloxy]sulfosuccinimide ester)) cross-linker and/or the protein is linked to the thiol-modified silica nanoparticles by a GMBS cross-linker.

6. The method of claim 1, wherein the substrate is glass, ceramic, silicon, or plastic.

7. The method of claim 1, wherein the substrate is an electrode, such as a silicon electrode.

8. The method of claim 1, wherein the substrate is a cell-contacting surface of a tissue culture vessel.

9. The method of claim 1, wherein the protein is a cell adhesion molecule, such as L1 polypeptide, an enzyme, or a binding reagent, such as an antibody or an antibody fragment.

10. (canceled)

11. The method of claim 1, further comprising linking the nanoparticles to the substrate using a suitable crosslinker to covalently link the nanoparticles to the substrate.

12. A kit comprising:
a dried protein-coated article, comprising:
a substrate;
silica nanoparticles linked to the substrate;
a protein linked to the silica nanoparticles; and
packaging containing the dried protein-coated article,
wherein none, or no more than 10%, 5%, or 1% of the activity of the protein is lost on storage of the dried, coated article in the packaging for at least 24 hours or at least 7 days at 25° C.

13. The kit of claim 12, wherein the dried, coated article is hermetically-sealed in the packaging.

14. The kit of claim 12, wherein the silica nanoparticles are linked to the protein and/or the substrate via a linker comprising a thioether bond.

15. The kit of claim 12, wherein the substrate is glass, ceramic, silicon, or plastic.

16. The kit of claim 12, wherein the substrate is an electrode, such as a silicon electrode or a cell-contacting surface of a tissue culture vessel.

17. The kit of claim 12, wherein the protein is a cell adhesion molecule, such as an L1 polypeptide, an enzyme, or a binding reagent, such as an antibody or an antibody fragment.

18. A method of treating a subject, comprising implanting into a patient a dried protein-coated article stored in a dry state for at least 24 hours, the article comprising:
a substrate;
silica nanoparticles linked to the substrate; and
a protein linked to the silica nanoparticles,
wherein none, or no more than 10%, 5%, or 1% of the activity of the protein is lost on storage of the dried, coated article in packaging for at least 24 hours or 7 days at 25° C.

19. The method of claim 18, wherein the silica nanoparticles are linked to the protein and/or the substrate via a linker comprising a thioether bond.

20. (canceled)

21. The method of claim 18, wherein the substrate is an electrode, such as a silicon electrode.

22. The method of claim 18, wherein the protein is a cell adhesion molecule, such as an L1 polypeptide, an enzyme, or a binding reagent, such as an antibody or an antibody fragment.
